(19) United States
(12) Patent Application Publication
(10) Pub. No.: US 2004/0210180 A1
(43) Pub. Date: Oct. 21, 2004

(54) Dialysis catheter system

(76) Inventor: Sanford D. Altman, North Miami Beach, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 32606-6669, US

(21) Appl. No.: 10/823,468

(22) Filed: Apr. 12, 2004

Related U.S. Application Data
(60) Provisional application No. 60/462,908, filed on Apr. 15, 2003.
(60) Provisional application No. 60/468,891, filed on May 8, 2003.

(51) Int. Cl. A61M 37/00; A61M 31/00; A61M 25/00; A61M 5/00

(52) U.S. Cl. 604/4.01; 604/6.16; 604/264

(57) ABSTRACT

The present invention provides a dialysis catheter that is designed to function in reverse-flow, having a dual lumen configuration. An embodiment of the present invention includes two lumen cooperatively configured in a co-axial design. The arterial lumen is circular or oval and extends beyond the termination of the venous lumen. The arterial lumen extracts the blood from the blood vessel for hemodialysis treatment. The venous lumen is also circular or oval. Terminating at a proximal point to the distal end of the arterial lumen, this configuration of the venous lumen aids in preventing recirculation. The venous lumen returns dialyzed blood back into the patient. The venous lumen can further include a plurality of apertures to aid in reducing the risk of fibrin sheath growth. In a method of use, the arterial lumen of the invention preferably resides within the right atrium with the venous lumen positioned within the superior vena cava.

### CROSS-REFERENCE TO A RELATED APPLICATION

This application claims the benefit of U.S. Provisional Application No. 60/462,908, filed Apr. 15, 2003 and U.S. Provisional Application No. 60/468,891, filed May 8, 2003.

### BACKGROUND OF INVENTION

It is estimated that the prevalence of chronic kidney disease in the United States population is 11% (roughly 19.2 million adult individuals) and increasing. The kidneys are organs which function to extract water and urea, mineral salts, toxins, and other waste products from the blood. Patients having one or both defective kidneys often require artificial “dialysis,” a procedure that simulates the function of the kidneys in cleaning wastes from the blood.

There are currently two forms of dialysis available: hemodialysis and peritoneal dialysis. Hemodialysis is a well-known method of providing renal (kidney) function by using a machine to clean wastes and extra fluids from blood and to re-circulate the cleansed blood back into the patient's body. In hemodialysis procedures, blood is withdrawn from the patient's body through an access to a dialysis machine, also commonly referred to as a kidney (or dialysis) machine. In the dialysis machine, toxins and other waste products diffuse through a semi-permeable membrane into a dialysis fluid closely matching the chemical composition of the blood. The filtered blood (i.e., blood with the waste products removed) is then returned to the patient's body. As can be appreciated, proper access to the patient's blood and transport of the blood to and from the dialysis machine for this extended period of time is critical to hemodialysis.

A hemodialysis access (or vascular access) is a large diameter, fast flowing conduit that is located just beneath the skin surface. The superficially located, large diameter, and fast flow conduit/access is typically punctured three times per week with two needles, wherein one needle removes blood from the patient's body and the second needle returns cleansed blood to the patient's body. The blood goes through the dialysis machine and through a special filter called a dialyzer. A patient can receive hemodialysis treatment through either a catheter, graft, or fistula.

A catheter can function as a temporary or permanent access, which consists of a tube placed directly into a large vein. With hemodialysis treatment, a catheter is connected directly to a dialysis machine and does not require the use of needles. The catheter may be a single tube with two separate lumens or two separate tubes. Generally, the length and diameter of the catheter will affect the catheter flow rates and pressures while performing dialysis.

Maintenance of a good access is a major cost of dialysis, which is the most common extracorporeal blood treatment, although other types of blood treatment are also used, for example passing of the blood through an absorption bed for removal of toxins and the like, hemoperfusion, and other forms of blood treatment.

Catheters which are implanted in the venous system of a patient for dialysis access or the like may develop a “fibrin sheath” on the outside of the catheter within the blood vessel, for example the jugular, subclavian, femoral veins or the vena cava. This fibrin sheath coats the outside of the catheter and can extend over the end thereof.

At the outflow port, this is generally not too serious a problem since the outflowing blood forces the fibrin sheath open easily. However, at the inflow port of the catheter, the sheath can act as a one way valve, collapsing with increasing negative pressure to seriously interfere with flow through the catheter. Upon such an occurrence, the blood flow through such a blood access catheter can occasionally be reversed for continuation of a desired medical procedure such as hemodialysis.

There are currently a wide variety of dual lumen catheters available for dialysis. These dual lumen catheters are available in a variety of configurations including the double-D configuration in which two catheters are placed side-by-side. Double-D catheters are presented either as two separate, and distinct, catheters residing within a single lumen or as a single catheter in which two lumens are separated by a shared wall.

In another example, a “circle C” catheter tube is available wherein the catheter tube has two coaxial lumens defined by a substantially circular outer wall member separated by a circular inner common support wall that joins the outer member. The outer lumen is substantially crescent-shaped in cross-section and can include a variety of apertures to allow for efficient fluid entry or discharge. The crescent-shaped lumen substantially surrounds the first lumen. The crescent-shaped lumen may be used to remove blood from the patient's body and the circular lumen may be used for returning blood to the patient. Alternatively, this device can be used in a reverse manner, with fluid withdrawal accomplished via the circular lumen and fluid return via the crescent-shaped lumen.

In yet another configuration, true coaxial dual lumen catheters are available. Co-axial dual lumen catheters have two lumens, wherein a first lumen resides within a second lumen such that the second lumen completely encompasses the first lumen.

Despite advances that have been made in providing vascular access for dialysis, there are a variety of problems associated with currently available catheters. For example, a significant problem with dialysis catheters is the risk of infection and clotting. The suction produced at the opening of a hemodialysis catheter can be occluded by intimal tissues (i.e., a fibrin sheath) within the blood vessel and result in clotting.

Hence, despite the availability of the above catheter devices, there is a continuing need for an improved dialysis catheter that decreases the potential for fibrin sheath formation and reduces the risk of infection while allowing for more effective dialysis.

### BRIEF SUMMARY

The present invention provides a unique catheter for use during dialysis. The invention pertains to a catheter designed to function primarily in “reverse-flow,” having a dual lumen configuration (i.e., co-axial, circle C, double D, and side-by-side configurations) in which the arterial lumen extends beyond the termination point of the venous lumen. According to the subject invention, the “arterial lumen” is utilized to remove blood from the patient's vasculature while the “venous lumen” is utilized to return treated blood to the patient.

The two lumens that form the reverse-flow catheter of the subject invention are positioned in use such that the terminal portion of the arterial lumen is in close proximity to the terminal portion of the venous lumen. In accordance with the present invention, the catheter can be formed such that the arterial lumen and venous lumen are situated side-by-side in what is commonly known as a “double-D” configuration or the arterial lumen is disposed within the venous lumen (i.e., co-axial configuration, “circle-C” configuration (having a crescent-shaped cross section)). In any position, according to the present invention, the arterial lumen extends beyond the termination point of the venous lumen.

In a preferred embodiment, the dual lumen catheter of the subject invention has lumens disposed one within the other in a co-axial configuration. The catheter includes a first lumen that is substantially circular in cross-section and which is defined by the inner surface of a tubular first wall and a second lumen that is also substantially circular or oval in cross-section, defined by the outer surface of the tubular first wall and inner surface of a second wall.

It is intended that the first lumen, defined by the tubular first wall, will be used as the arterial lumen (also referred to herein as the arterial line) to withdraw fluid from the patient. It is also intended that the second lumen, defined by the second wall, will be used as the venous lumen (also referred to herein as the venous line) to return cleansed fluid (i.e., blood) to the patient.

According to the present invention, dual-lumen catheters are designed to function in a reverse-flow manner wherein cleansed blood returns through the second/venous lumen, which terminates at a point prior to the terminal point of the first lumen. The return of blood through the second/venous lumen allows for high flow/high pressure return of blood proximal to the first/arterial lumen, thereby preventing or reducing the likelihood of fibrin sheath forming around the distal end of the first/arterial lumen. This reduction in fibrin sheath formation may allow for improved catheter flow rates for longer periods of time than those generally observed with conventional catheters.

The second/venous lumen of the subject invention is of shorter length than typical venous lumen of non-reverse flow catheters. As a result of terminating prior to the distal end of an arterial line, which results in a decrease in length of the venous lumen, the pressure within the venous lumen of the subject invention is less than the pressure commonly seen within the venous lumen of non-reverse flow catheters of similar diameter size.

The catheter of the subject invention is normally placed in a large vein of a patient, in the direction of blood flow in the vein. For example, a dual-lumen, reverse-flow catheter can be placed in the jugular vein, the subclavian vein, brachieocephalic vein, hepatic vein, femoral vein, or vena cava, in accordance with the subject invention. The blood is drawn from the patient through an aperture at the distal end of the first/arterial lumen and returned through at least one aperture at the distal end of the second/venous lumen.

In one embodiment, the distal end of the venous lumen has one aperture located at the terminal point of the venous lumen and surrounding the arterial line. Such an aperture, according to the subject invention, allows fluid flow from the venous line to completely encircle/bath the segment of the arterial line that extends beyond the terminal point, distal end of the venous line. In another embodiment, the distal end of the venous lumen has a plurality of apertures surrounding the arterial catheter. In yet another embodiment, the terminal point of the distal end of the venous lumen is sealed (i.e., fused to the arterial line, wherein fluid outflow through the venous lumen is accomplished through the plurality of apertures located on the distal end of the venous lumen. In certain embodiments, the apertures are in the shape of circles, ovals, and/or slits along the distal end of the venous catheter. All of the designs disclosed above will help in reducing fibrin sheath growth around the distal end of the arterial lumen.

A catheter of the subject invention can be introduced to a patient either alone or with the aid of a guide wire. The coaxial design of the subject invention is particularly advantageous in over the wire introduction of the catheter into the desired vein. In certain embodiments, the subject catheter can be used with or without a fixed hub to allow for antegrade or retrograde tunneling for introducing the catheter to a patient.

In another embodiment, the surface of the arterial and/or venous lumen can be treated and/or fabricated with substances known to aid in decreasing fibrin sheath formation and/or decrease the risk of infection. In a related embodiment, the surface of the arterial and/or venous lumen is textured so as to prevent fibrin and/or thrombin formation about the catheter.

In other embodiments, the catheter of the subject invention includes elongate ridges or spokes located between the inner surface of the venous lumen and the outer surface of the arterial lumen. Such ridges or spokes are provided to maintain an aperture of the venous lumen through which fluid (i.e., blood) can properly flow into the patient.

### BRIEF DESCRIPTION OF THE DRAWINGS

FIGS. 1A-1D show perspective, side views of embodiments of the catheter of the present invention.

FIGS. 2A-2B show a perspective, side view of an embodiment of the catheter of the present invention, wherein the arterial lumen is separable from the venous lumen.

FIG. 3 shows a perspective, side view of an embodiment of the catheter of the present invention having a tapered distal end of the arterial lumen.

FIG. 4 shows a perspective, side view of an embodiment of the catheter of the present invention having a tapered distal end of the venous lumen.

FIG. 5 shows a perspective, side view of a catheter of the present invention having more than one aperture on the venous lumen.

FIG. 6 shows a perspective, side view of an embodiment of the catheter of the present invention having a plurality of slits on the venous lumen.

FIG. 7 shows a cross-sectional view of an embodiment of the catheter of the present invention having ridges therein.

FIG. 8 shows a cross-sectional view of an embodiment of the catheter of the present invention having spokes therein.

### DETAILED DISCLOSURE

The present invention provides a unique catheter for use during dialysis, in particular during hemodialysis. The invention pertains to a catheter primarily designed to function in reverse-flow, having a dual lumen configuration (i.e., co-axial, circle C, double D, and side-by-side configurations) in which the arterial lumen extends beyond the termination point of the venous lumen.

The catheter of the subject invention preferably functions in “reverse-flow” to aid in reducing fibrin sheath and/or thrombosis formation and to provide fluid return at effective flow rates and at lower pressures than those typically observed with traditional catheters designed to function in a non-reverse flow manner.

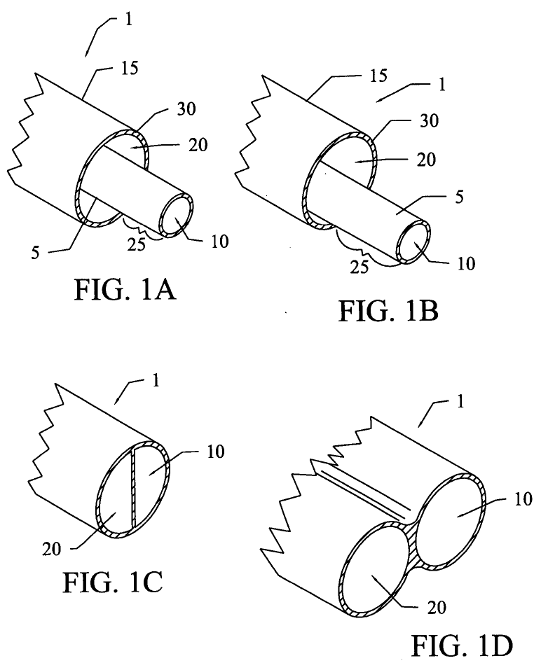

According to the present invention, dual-lumen, reverse-flow catheters can be formulated in a wide variety of configurations (see FIGS. 1A-1D). The dual-lumen catheter of the present invention can be formulated in the following configurations including, but not limited to, a co-axial configuration (see FIG. 1A); a circle C configuration (see FIG. 1B); a double-D configuration (see FIG. 1C); and a side-by-side configuration (see FIG. 1D). In accordance with the present invention, the termination point of the arterial lumen extends beyond the termination point of the venous lumen in any configuration.

In a preferred embodiment, the lumens are disposed one within the other in a co-axial configuration, as shown in FIGS. 1A and 1B. The catheter 1 includes a first (arterial) lumen 10 that is substantially circular or oval in cross-section, which is defined by the inner surface of a tubular first wall 5, and a second (venous) lumen 20 that is also substantially circular or oval in cross-section, which is defined by the outer surface of the tubular first wall and inner surface of a tubular second wall 15.

The arterial lumen 10 preferably extends beyond the termination point 30 of the venous lumen 20. By extending the arterial lumen 10 beyond the termination point 30 of the venous lumen 20, the present invention allows for returned blood to bathe the area surrounding the distal portion 25 of the arterial lumen and thus reduces fibrin sheath formation around the distal portion 25 of the arterial lumen 10.It is intended that the arterial lumen 10, defined by the tubular first wall 5, will be used to withdraw fluid from the patient. It is also intended that the venous lumen 20, defined by the tubular second wall 15, will be used to return cleansed fluid to the patient.

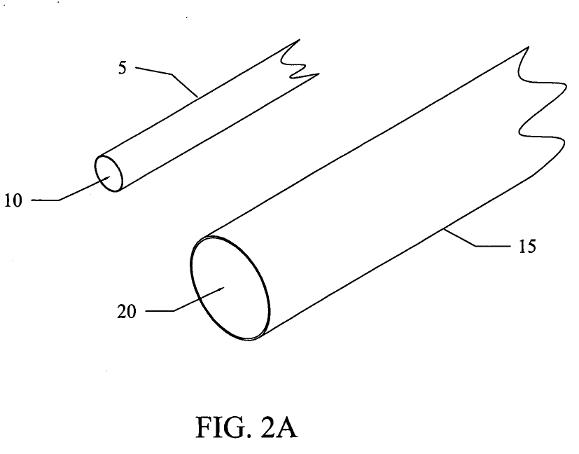

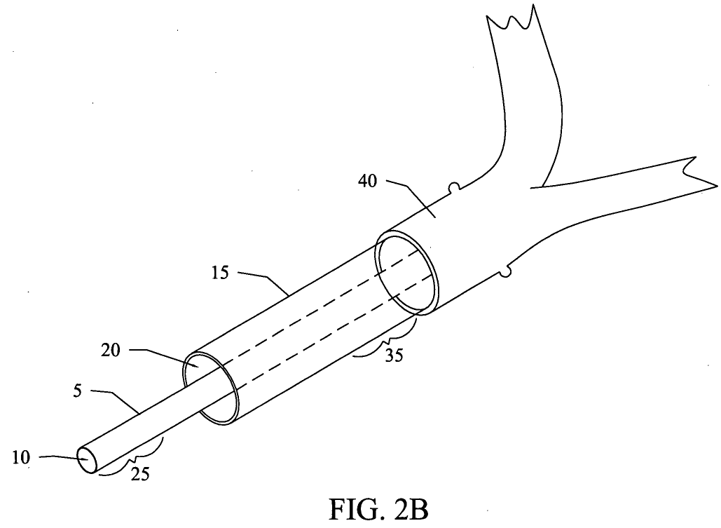

In accordance with the present invention, as illustrated in FIGS. 2A and 2B, the arterial lumen 10 can be separated from the venous lumen 20 to enable removal and replacement of the arterial lumen. As illustrated in FIG. 2B, the proximal end 35 of the catheter 1 of the subject invention, which includes co-axially configured, separable arterial and venous lumens, is connected to a fluid-conveying, removable, hollow hub assembly 40. According to the subject invention, the catheter 1 can be disconnected from the hub assembly 40 in order to remove the arterial lumen 10 from the venous lumen 20 (see FIG. 2A) for repair and/or replacement to ensure optimal dialysis function.

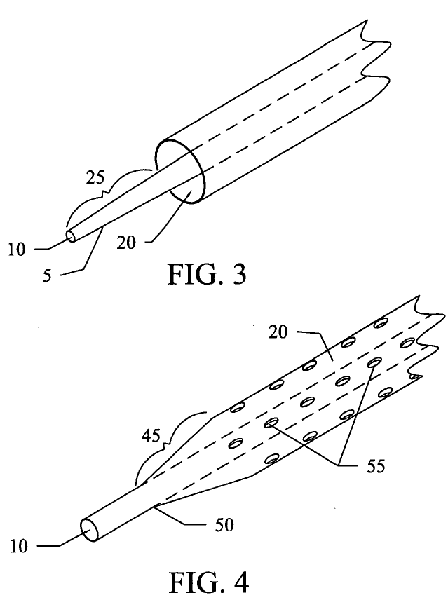

The dual-lumen catheter 1 of the subject invention is configured to be operatively coupled between a patient and a dialysis machine for hemodialysis treatment. In certain embodiments, the catheter includes two corresponding, co-axial conduits. The arterial lumen 10, which extends beyond the length of the venous lumen 20, can be tapered at the distal end 25 (see FIG. 3) for ease of introduction into the patient's blood vessel (i.e., introduction over a guide wire). Alternatively, as illustrated in FIG. 4, the termination point 30 of the venous lumen can be fused to (as illustrated in FIG. 4) or separate (as seen in FIG. 1A) from the tubular first wall 5 of the arterial lumen and can be tapered at the distal end 45 (also for ease of introduction into the patient's blood vessel). The arterial lumen extracts blood from the patient's blood vessel and delivers the blood to the dialysis machine for treatment. The arterial lumen can include one or more apertures for extracting blood.

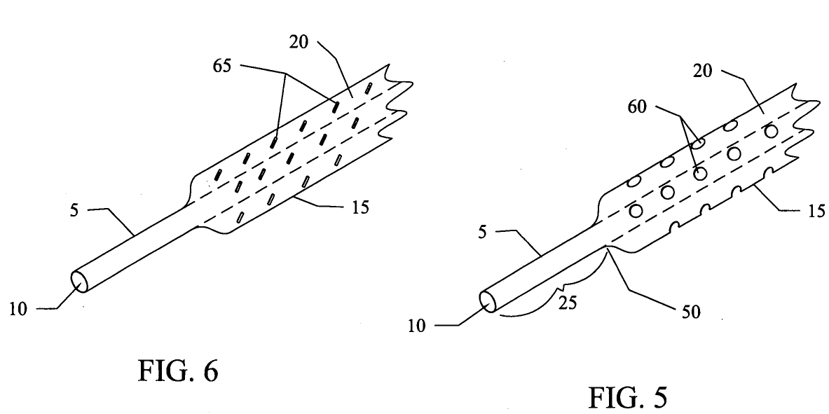

The venous lumen delivers the treated blood back into the patient's body. The point at which the venous lumen terminates is of sufficient distance from the distal end of the arterial lumen to prevent significant recirculation of dialyzed blood. The venous lumen 20 can include one ore more apertures for returning dialyzed blood to the patient (see FIGS. 4-6). In a preferred embodiment, the distal end 45 of the venous lumen is fused 50 onto the outer surface of the first tubular wall 5 of the arterial lumen 10 . As illustrated in FIGS. 4 and 5, the outer surface of the second tubular wall 15 of the venous lumen 20 can include a plurality of apertures in the shape of circles 55 or ovals 60 to provide return of fluid to the patient's body. Alternatively, as illustrated in FIG. 6, the outer surface of the second tubular wall 15 of the venous lumen 20 can include a plurality of apertures in the form of slits 65 to disseminate treated fluid back to the patient's body.

In one embodiment, the arterial lumen extends about 5-10 cm from the most distal end of the venous lumen. Preferably, the arterial lumen extends about 6-8 cm from the most distal end of the venous lumen. Most preferably, the arterial lumen extends 7 cm from the most distal end of the venous lumen. The venous lumen of the subject invention is of shorter length than typical venous lumen of non-reverse flow catheters.

In accordance with the present invention, the return of blood through the venous lumen allows for high flow/high pressure return of blood proximal to the arterial lumen thereby preventing or reducing the likelihood of fibrin sheath forming around the distal end of the arterial lumen. Reduction in fibrin sheath formation can allow for improved catheter flow and longevity of catheter use. In addition, the decrease in length of the venous venous lumen decreases the pressure within the venous lumen of the subject invention. Thus, the catheter of the subject invention allows for venous lumen pressure that is less than the pressure commonly seen within the venous lumen of non-reverse flow catheters of similar diameter size. Decreased venous lumen pressure allows for more comfortable and less traumatic hemodialysis procedures for the patient.

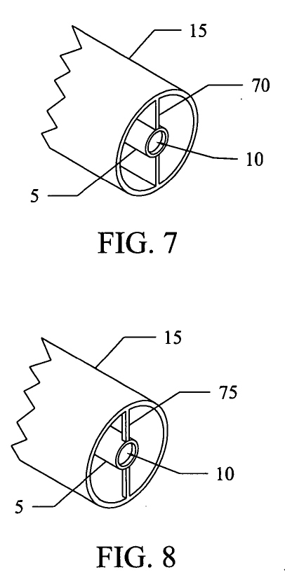

In another embodiment of the present invention, at least one elongate ridge 70 (see FIG. 7) that runs substantially the length of the catheter and is attached between the outer surface of the tubular first wall 5 of the arterial lumen 10 and the inner surface of the tubular second wall 15 of the venous lumen 20. The ridges 70 secure the position of the venous lumen 20 with respect to the arterial lumen 10. In addition, the ridges 70 provide a means for maintaining an aperture of the venous lumen 20 through which returning fluid can be provided to the patient's body.

In another embodiment of the present invention, as illustrated in FIG. 8, at least one spoke 75 can be provided between the outer surface of the tubular first wall 5 of the arterial lumen 10 and the inner surface of the tubular second wall 15 of the venous lumen 20 to maintain an aperture of the venous lumen 20 (i.e., an aperture located at the termination point of the distal end of a venous lumen). In certain embodiments, a plurality of spokes 75 is provided in intermittent positions, equidistant from each other, along the circumference of the termination point of a venous lumen, wherein the spokes are situated between the inner surface of the tubular second wall of the venous lumen and the outer surface of the tubular first wall of the arterial lumen. In other embodiments, a plurality of spokes 75 is located along the length of the venous lumen, wherein the spokes are located between the inner surface of the tubular second wall of the venous lumen and the outer surface of the tubular first wall of the arterial lumen.

Contemplated patient's blood vessels in which the catheter of the subject invention can be presented include, but are not limited to, the jugular vein, the subclavian vein, brachiocephalic vein, hepatic vein, femoral vein, and the inferior vena cava. In a preferred embodiment, the dual-lumen, reverse-flow catheter of the invention is introduced through the jugular vein.

The catheter, including the arterial lumen, venous lumen, and any elongate ridges and/or spokes, can be manufactured from same or different materials. According to the subject invention, the catheter, arterial lumen, and/or venous lumen are typically made from substantially flexible materials such as, but not limited to, thermoplastics, high performance engineering resins, polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), and silicone. Additional material may be incorporated into the wall of the catheter to reduce catheter kinking. Examples of contemplated materials for incorporation into the wall of the catheter to reduce kinking include, but are not limited to, metals, stainless steel, nickel alloys, nickel-titanium alloys, or other alloys. In preferred embodiments, the catheter, arterial lumen, and/or venous lumen are composed of polyethylene or polyvinyl chloride.

According to the subject invention, the catheter, including arterial lumen and venous lumen, can be manufactured to include antithrombin agents, antifibrin agents, anticoagulants, and/or antimicrobial agents. In certain embodiments, such agents are combined with materials described above that are used to make a catheter of the subject invention. For example, the catheter, arterial lumen, and/or venous lumen of a catheter of the subject invention can be composed of polyethylene or polyvinyl chloride that is impregnated with an antifibrin agent, an antithrombin agent, an anticoagulant, and/or an antimicrobial agent. In other embodiments, the surface of a catheter, arterial lumen, and/or venous lumen of the subject invention can include a layer or coating consisting of an antifibrin agent, an antithrombin agent, an anticoagulant, and/or an antimicrobial agent.

The surface of a catheter of the subject invention, including the surface of the arterial lumen and/or venous lumen, can be constructed so as to include a textured surface to prevent fibrin and/or thrombin formation about the catheter. For example, the surface at the distal end of an arterial lumen of the invention can include microscopic nubs that are coated with an antifibrin and/or antithrombin agent to prevent fibrin and/or thrombin formation about the termination point of the catheter, in particular the termination point of the arterial lumen.

In a method of use, after appropriately preparing and anesthetizing a patent, a small incision is made to the patient's blood vessel (i.e., venetomy in the jugular vein). The catheter of the subject invention is normally placed in patient's blood vessel, in the direction of blood flow. Blood is then drawn from the patient through an aperture at the distal end of the first/arterial lumen and returned through at least one aperture at the distal end of the second/venous lumen.

In a preferred embodiment, a small incision is made in a large vein (i.e., jugular vein). The catheter of the subject invention is then inserted through the incision so that the first/arterial lumen of a catheter is placed within the right atrium of the patient's heart and the second/venous lumen is placed within the superior or inferior vena cava.

In another embodiment, after appropriately preparing and anesthetizing a patient and making a small incision to the patient's blood vessel, a guide wire is first introduced into the blood vessel in the direction of the blood flow. The vein entrance site is then dilated to the appropriate diameter to accommodate the reverse flow catheter. Thereafter, the distal end of the arterial lumen is fed through the incision over the guide wire allowing the catheter to be introduced into the body without the use of an introduction sheath. In a preferred embodiment, the arterial lumen is fed over the guide wire until it is placed within the right atrium of the patient's heart and the venous lumen is placed within the superior or inferior vena cava. Once the catheter is positioned as desired in the patient, the guide wire is removed and the catheter flushed with an appropriate solution known to the skilled artisan (i.e., saline solution).

In certain methods of use, the catheter of the subject invention can include a hollow hub assembly for use in antegrade tunneling or can be introduced to a patient without a hollow hub assembly so as to enable retrograde tunneling.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

I claim:

1. A dual-lumen, reverse-flow catheter comprising an arterial lumen having an inner surface, an outer surface, a distal end, and a proximal end; and a venous lumen having an inner surface, an outer surface, a distal end, and a proximal end, wherein the distal end of the arterial lumen extends beyond the distal end of the venous lumen, wherein the distal end of the arterial lumen comprises one aperture and the distal end of the venous lumen comprises at least one aperture.

2. The catheter of claim 1, wherein the arterial lumen is disposed within the venous lumen in a co-axial configuration.

3. The catheter of claim 2, wherein the distal end of the arterial lumen extends between 5-10 cm beyond the distal end of the venous lumen.

4. The catheter of claim 3, wherein the distal end of the arterial lumen extends between 6-8 cm beyond the distal end of the venous lumen.

5. The catheter of claim 4, wherein the distal end of the arterial lumen extends 7 cm beyond the distal end of the venous lumen.

6. The catheter of claim 2, wherein the distal end of the venous lumen is tapered.

7. The catheter of claim 2, wherein the distal end of the arterial lumen is tapered.

8. The catheter of claim 2, wherein the distal end of the venous lumen is fused onto the outer surface of the arterial lumen.

9. The catheter of claim 2, wherein at least one elongate ridge that runs substantially along the length of the catheter is attached between the outer surface of the arterial lumen and the inner surface of the venous lumen.

10. The catheter of claim 2, wherein at least one spoke is attached between the outer surface of the arterial lumen and the inner surface of the venous lumen.

11. The catheter of claim 1, wherein the arterial lumen is disposed within the venous lumen in a circle-C configuration.

12. The catheter of claim 11, wherein the distal end of the arterial lumen extends between 5-10 cm beyond the distal end of the venous lumen.

13. The catheter of claim 12, wherein the distal end of the arterial lumen extends between 6-8 cm beyond the distal end of the venous lumen.

14. The catheter of claim 13, wherein the distal end of the arterial lumen extends 7 cm beyond the distal end of the venous lumen.

15. The catheter of claim 11, wherein the distal end of the venous lumen is tapered.

16. The catheter of claim 11, wherein the distal end of the arterial lumen is tapered.

17. The catheter of claim 11, wherein the distal end of the venous lumen is fused onto the outer surface of the arterial lumen.

18. The catheter of claim 11, wherein at least one elongate ridge that runs substantially along the length of the catheter is attached between the outer surface of the arterial lumen and the inner surface of the venous lumen.

19. The catheter of claim 11, wherein at least one spoke is attached between the outer surface of the arterial lumen and the inner surface of the venous lumen.

20. The catheter of claim 1, wherein the arterial lumen and the venous lumen are positioned in a double-D configuration.

21. The catheter of claim 20, wherein the distal end of the arterial lumen extends between 5-10 cm beyond the distal end of the venous lumen.

22. The catheter of claim 21, wherein the distal end of the arterial lumen extends between 6-8 cm beyond the distal end of the venous lumen.

23. The catheter of claim 22, wherein the distal end of the arterial lumen extends 7 cm beyond the distal end of the venous lumen.

24. The catheter of claim 1, wherein the distal end of the venous lumen comprises a plurality of apertures.

25. The catheter of claim 24, wherein the apertures have a cross-sectional shape selected from the group consisting of circular, oval, or slits.

26. The catheter of claim 1, wherein the catheter includes an agent selected from the group consisting of: antifibrin agents, antithrombin agents, anticoagulant agents, and antimicrobial agents.

27. The catheter of claim 1, wherein the catheter is made from a substance selected from the group consisting of: thermoplastics, high performance engineering resins, polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), and silicone.

28. The catheter of claim 27, wherein additional substances for reducing kinking are included in making the catheter, wherein said additional substance is selected from the group consisting of: metals, stainless steel, nickel alloys, nickel-titanium alloys, or other alloys.

29. The catheter of claim 1, further comprising a hollow hub to which the catheter is connected, wherein the venous lumen is separable from the arterial lumen.

30. The catheter of claim 1, wherein the catheter is inserted into a patient having a right atrium and vena cava, wherein after insertion, the arterial lumen is positioned in the right atrium and the venous lumen is positioned in the vena cava.

31. A method for treating blood, said method comprising the steps of:
a) making an incision to a blood vessel; and
b) inserting into the blood vessel, in the direction of blood flow, a dual-lumen, reverse-flow catheter comprising an arterial lumen having an inner surface, an outer surface, a distal end, and a proximal end; and a venous lumen having an inner surface, an outer surface, a distal end, and a proximal end, wherein the distal end of the arterial lumen extends beyond the distal end of the venous lumen, wherein the distal end of the arterial lumen comprises one aperture and the distal end of the venous lumen comprises at least one aperture, wherein blood from the blood vessel is drawn through the aperture at the distal end of the arterial lumen;
c) treating the drawn blood; and
d) returning the treated blood to the blood vessel through the at least one aperture at the distal end of the venous lumen.

32. The method of claim 31, further comprising the step of placing the catheter such that the arterial lumen is situated in a right atrium and the venous lumen is situated in a superior vena cava.

33. The method of claim 31, further comprising the step of inserting a guide wire into the incision and feeding the arterial lumen into the incision over the guide wire into the blood vessel.

34. The method of claim 31, wherein the catheter includes a removable, hollow hub to which the catheter is connected and the venous lumen is separable from the arterial lumen, further comprising the steps of removing the catheter from the blood vessel and replacing the arterial lumen with a new arterial lumen.

35. The method of claim 31, wherein the blood vessel is selected from the group consisting of jugular vein, hepatic vein, femoral vein, and inferior vena cava.
